(19) United States
(12) Patent Application Publication
(10) Pub. No.: US 2008/0248540 A1
(43) Pub. Date: Oct. 9, 2008

(54) METHODS OF PRODUCING BUTANOL

(75) Inventor: Shang-Tian Yang, Dublin, OH (US)

Correspondence Address: DINSMORE & SHOHL LLP, ONE DAYTON CENTRE, ONE SOUTH MAIN STREET, SUITE 1300, DAYTON, OH, 45402-2023, US

(73) Assignee: THE OHIO STATE UNIVERSITY, Columbus, OH (US)

(21) Appl. No.: 12/062,091

(22) Filed: Apr. 3, 2008

Related U.S. Application Data
(60) Provisional application No. 60/909,729, filed on Apr. 3, 2007.

(51) Int. Cl. C12P 7/16

(52) U.S. Cl. 435/160

(57) ABSTRACT

A method of producing butanol from carbohydrates is provided. A feedstock comprising a carbohydrate source is fermented in the presence of bacteria to produce butyric acid and hydrogen. The butyric acid is then hydrogenated in the presence of a catalyst to produce butanol.

### CROSS REFERENCE TO RELATED APPLICATIONS

This application claims the benefit of U.S. Provisional Application Ser. No. 60/909,729 filed Apr. 3, 2007.

### TECHNICAL FIELD

The present invention is generally directed to methods of producing butanol, and more specifically, to methods of producing butanol from biomass via fermentation and catalytic hydrogenation.

### BACKGROUND

Fermentation processes using microorganisms provide a promising path for converting biomass and agricultural wastes into chemicals and fuels. There are abundant low-value agricultural commodities and food processing byproducts/wastes that require proper disposal to avoid pollution problems. In the corn refining industry, more than 22% of the estimated 11.8 billion bushels (˜300 million metric tons) of corn annually produced in the U.S. are processed to produce high-fructose-corn-syrup, dextrose, starch, and fuel alcohol. Also, there is an increasing commercial interest in developing a micron milling process for the production of corn protein isolate (CPI) from defatted corn germ. The main byproduct from this process is corn starch, which must be properly converted into marketable products, such as organic acids and alcohols, in order to avoid the high waste treatment costs (due to its high BOD content). In addition to starch, it is also desirable to utilize the abundant pentoses present in the hemicelluloses found in corn fibers, corn cobs, and many other agricultural crops and plant biomasses.

As crude oil prices have risen, biobutanol has become an attractive transportation fuel. Butanol has many characteristics that make it a better fuel than ethanol, now produced from corn and sugar cane. As a biofuel, butanol has the following advantages over ethanol: (a) butanol has 30% more Btu per gallon; (b) butanol is less evaporative/explosive with a Reid vapor pressure (RVP) 7.5 times lower than ethanol; (c) butanol is safer than ethanol because of its higher flash point and lower vapor pressure; (d) butanol has a higher octane rating; and (e) butanol is more miscible with gasoline and diesel fuel but less miscible with water. Butanol offers a safer fuel that can be dispersed through existing pipelines and filling stations. However, butanol is currently almost exclusively produced via petrochemical routes. Butanol finds use in industrial applications in solvents, rubber monomers and break fluids. Butanol is also utilized in the food and cosmetic industries as an extractant, but there are concerns of carcinogenic effects associated with the petroleum-based butanol.

Acetone-butanol-ethanol fermentation (ABE fermentation) with the strict anaerobic bacterium Clostridium acetobutylicum was once a widely used industrial fermentation process. However, since the 1950's, industrial ABE fermentation has declined continuously with the last commercial fermentation plant closing in the 1980's. In a typical ABE fermentation, butyric and acetic acids are produced first by C. acetobutylicum. The culture then undergoes a metabolic shift and solvents (butanol, acetone, and ethanol) are formed. Increasing butyric acid concentration to >2 g/L and decreasing the pH to <5 usually are required for the induction of a metabolic shift from acidogenesis to solventogenesis. However, the actual fermentation is quite complicated and difficult to control. In conventional ABE fermentations, the butanol yield from glucose is low, typically at ˜15% (w/w) and rarely exceeds 25%. The production of butanol is also limited by severe product inhibition, resulting in a low reactor productivity of usually less than 0.5 g/L·h and a low final butanol concentration of less than 15 g/L. The low reactor productivity, butanol yield, and final butanol concentration make traditional butanol production from biomass by ABE fermentation uneconomical.

The fermentation route is not competitive with petroleum-based solvent synthesis methods unless new technologies overcoming all these limitations can be developed. There have been numerous attempts to improve butanol production in ABE fermentation via metabolic engineering of the fermentation microorganisms and process engineering to alleviate inhibition caused by butanol and facilitate product recovery. Despite all these efforts, there has been little progress and it is apparent that butanol produced via ABE fermentation is not likely to become economically competitive as a biofuel in the foreseeable future. A new process for the production of biobutanol from biomass is thus needed.

### SUMMARY

According to one embodiment of the present invention, a method of producing butanol from carbohydrates is provided. A feedstock comprising a carbohydrate source is fermented in the presence of bacteria to produce butyric acid and hydrogen. The butyric acid is then hydrogenated in the presence of a catalyst to produce butanol.

### BRIEF DESCRIPTION OF THE DRAWINGS

FIG. 1 is a flow chart illustrating one embodiment of the fermentation-catalytic hydrogenation process for biobutanol production.

FIG. 2 is a flow chart illustrating one embodiment of the fermentation-catalytic hydrogenolysis process for biobutanol production.

### DETAILED DESCRIPTION

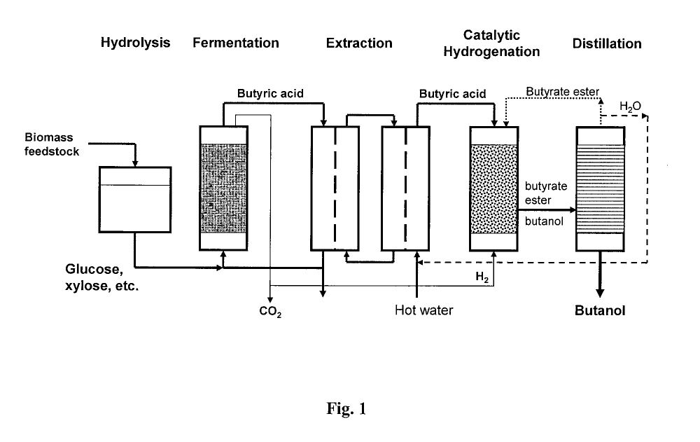

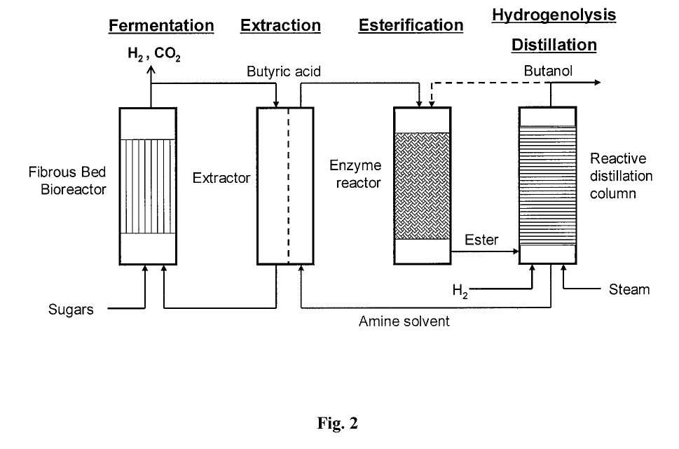

Instead of producing butanol from biomass directly by fermentation, which is inherently difficult and uneconomical, a novel process is provided which first converts biomass or fermentable carbohydrates to butyric acid by fermentation with bacteria, and then converts butyric acid and hydrogen to butanol by catalytic hydrogenation or hydrogenolysis of butyrate ester. In some embodiments the butyric-acid producing bacteria are aerobic bacteria, and in other embodiments, the butyric-acid producing bacteria are anaerobic bacteria. FIG. 1 illustrates a flowchart of one embodiment of the process with hydrogenation. FIG. 2 illustrates a flowchart of one embodiment of the process with hydrogenolysis.

A biomass feedstock with fermentable carbohydrates is fed to a bioreactor, such as a fibrous bed bioreactor as disclosed in U.S. Pat. No. 5,563,069, for butyric acid fermentation by butyric acid producing bacteria, such as Clostridium tyrobutyricum at mildly elevated temperatures, typically ˜37° C. The fermentation process may also utilize other butyric acid producing bacteria, as the specific recitation of Clostridium tyrobutyricum is not meant to limit the scope of the invention. Other suitable bacteria include C. butyricum, C. beijerinckii, C. populeti and C. thermobutyricum. In preferred embodiments, butyric acid fermentation utilizing mutants of Clostridium tyrobutyricum ATCC 25755 obtained from inactivating the chromosomal ack gene, encoding acetate kinase, and adaptation in a fibrous bed bioreactor showed significantly improved butyric acid production with a high butyric acid yield of up to 48% (w/w), final concentration of up to 80 g/L, and high productivity (>2 g/L·h) from glucose and xylose. Hydrogen and carbon dioxide are also produced in the fermentation of carbohydrates in the presence of Clostridium tyrobutyricum. The fermentation can be carried out in either batch, fed-batch, or continuous mode to optimize yield and lower production cost. Acetic acid is a byproduct from the fermentation, but its concentration is relatively low and its presence does not adversely affect the subsequent catalytic hydrogenation reaction.

Hydrogen produced by the fermentation process may be separated from carbon dioxide and then utilized in the hydrogenation process after compression. The amount of hydrogen produced in the butyric acid fermentation is sufficient for the production of butanol in the catalytic hydrogenation process. Thus, hydrogen required for the hydrogenation process may be obtained solely from the hydrogen produced in the fermentation process. However, particular embodiments of the invention may or may not utilize only the hydrogen produced in the fermentation process, as additional sources of hydrogen may also be employed in the hydrogenation process.

The butyric acid present in the fermentation broth is recovered and purified by extraction using an aliphatic amine, such as Alamine 336, or other water-immiscible solvents. When the fermentation is coupled with the extraction, the resulting extractive fermentation process produces a much higher butyrate concentration at a higher productivity and purity. The butyric acid present in the solvent is stripped with hot water or steam in a separate extractor and the partially purified (and concentrated) butyric acid is fed into the catalytic hydrogenation reactor for butanol production. Additionally, the butyric acid can also be converted to butyrate ester with an alcohol (preferably butanol) and then fed to the catalytic hydrogenation reactor for butanol production.

Carboxylic acids are catalytically converted to corresponding alcohols by hydrogenation with metal oxides catalysts under elevated pressures and temperatures. Catalytic hydrogenation can achieve high selectivity (over 95%) and conversion (>70%) at a relatively short reaction time (a few hours). The reaction converts the acids to alcohols and their esters as byproducts. This process can be used with acetic acid (ethanol), propionic acid (propanol), and many fatty acids (fatty acid esters). In general, the hydrogenation reaction works faster and with higher yields for fatty acids with longer chain lengths. The product alcohol can be separated from unreacted carboxylic acid and the byproducts (water and esters) by distillation. With 100% conversion, the theoretical yield of butanol from butyric acid in the catalytic hydrogenation is 83% (w/w).

After catalytic hydrogenation or hydrogenolysis, the mixtures (butanol, butyrate ester, water, etc.) can be separated by conventional distillation. Butanol, which has a low vapor pressure and low water solubility, is separated and removed from the bottom of the distillation column. Butyrate ester and water are separated and removed from the top of the distillation column and can be recycled as shown in FIG. 1.

### Materials Balance Based on Reaction Stoichiometry:

1. Butyric acid fermentation without acetic acid formation:

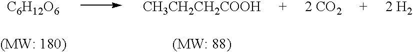

2. Hydrogenation of butyric acid to butanol:

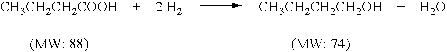

### Overall Reaction:

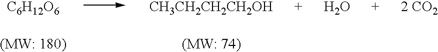

A more detailed description of the fermentation and the hydrogenation steps of the process follows.

Butyric Acid Fermentation

Plant biomass represents a useful and valuable resource as a fermentation substrate for highly valuable organic fuels and chemicals. Plant biomass generally consists of ˜25% lignin and ˜75% carbohydrate polymers including cellulose and hemicellulose. The latter represents one fifth to one half of the total carbohydrates in the biomass. Cellulose is a heteropolymer of hexose and pentose sugars, with glucose and xylose as two major constituents. While fermentation has been widely used to produce various fuels and chemicals, many of the current industrial fermentation processes cannot use pentose sugars as the carbon source. However, for economic use of plant biomass in industrial fermentations, it is important to convert all the sugars derived from plant biomass into the final products. Therefore, effective utilization of xylose and other pentoses is important to the bioconversion of hemicellulose.

Similarly, butyric acid can also be produced from sugars present in or derived from other plant biomasses such as casava, corn cob, wheat bran, rice straw, sugarcane bagasse, and any biomass containing starch, cellulose, hemicellulose, and other sugars. The following example demonstrates that low-value corn refining byproducts can be efficiently used for butyrate production.

### EXAMPLE 1

### Fermentation

Acid Hydrolysis of Corn Fiber. Fresh corn fibers were dried at 60° C. for 12 hours, and then analyzed for moisture, ash and organic contents. The carbohydrate contents of corn fibers were analyzed after complete hydrolysis with acid. The conditions of acid hydrolysis were studied to achieve a maximum release of the component sugars for fermentation. The dried fibers were mixed with dilute acid, either hydrochloric acid or sulfuric acid (9 ml acid solution per gram solid) at various concentrations (final concentrations: 0.1-0.5 M) and autoclaved at 121° C., 15 psig for 15-60 min. In preparing the corn fiber hydrolysate (CFH) for this example, dried corn fibers were hydrolyzed with 0.25 N HCl at 121° C. for 45 min. Insoluble materials were removed by filtration and the remaining hydrolysate was stored at 4° C. The composition of the CFH was analyzed with high performance liquid chromatography (HPLC).

Culture and Media. The acidogenic bacterium C. tyrobutyricum ATCC 25755 was cultured in a synthetic medium with either glucose or xylose as the substrate. The stock culture was kept in serum bottles under anaerobic conditions at 4° C. Concentrated substrates containing 30 g/L of xylose or 20-50 g/L of glucose were used. A sugar mixture containing 15 g/L xylose and 15 g/L glucose as the growth substrates was also prepared. The CFH from above contained 22.6 g/L xylose, 29.2 g/L glucose, 11.7 g/L arabinose, 2.8 g/L acetic acid and 0.5 g/L lactic acid, and was supplemented with nutrients from corn steep liquor (CSL), which was obtained from a corn wet-milling plant and was stored at 4° C. The CSL also contained about 53.6 g/L glucose, 18.2 g/L fructose, and 50.8 g/L lactic acid. It was diluted using equal parts water before use. To prepare for fermentation, 500 ml of the diluted CSL was mixed with 1200 ml CFH and neutralized to pH 6 with NH₄OH. All the media were sterilized by autoclaving at 121° C., 15 psig, for 30 min.

Fermentation in a Fibrous Bed Bioreactor. The fibrous bed bioreactor comprised a glass column packed with a spiral wound cotton towel and had a working volume of ˜480 ml. Before use, the bioreactor was autoclaved for 30 min at 121° C., held overnight and then autoclaved again for another 30 min for complete sterilization. The column reactor was aseptically connected to a sterile 5-L stirred-tank fermentor through a recirculation loop. The entire reactor system contained ˜2 L of the medium. Anaerobiosis was reached by sparging the medium with N₂. In this example, the reactor temperature was maintained at 37° C., agitation at 150 rpm, and pH controlled at 6.0 by adding NH₄OH or 6 N HCl. To start the fermentation, ˜100 ml of cell suspension of the bacteria in serum bottles were inoculated into the fermentor and allowed to grow for 3 days until the cell concentration reached an optical density (OD₆₂₀ₙₘ) of ˜4.0. Cell immobilization was then carried out by circulating the fermentation broth through the fibrous bed at a pumping rate of ˜25 ml/min to allow cells to attach and be immobilized onto the fibrous matrix. After about 36-48 hours of continuous circulation, most of the cells were immobilized and no change in cell density in the medium could be identified. The medium circulation rate was then increased to ˜100 ml/min for the subsequent fermentation. The reactor was operated at a repeated batch mode during the start-up period to increase the cell density in the fibrous bed to a stable, high level (˜70 g/L). In the repeated batch fermentation, the fermentation broth in the fermentor was replaced with fresh medium to start a new batch but the immobilized cells in the bioreactor were allowed for continued growth batch after batch. To study the fermentation kinetics, the broth in the fermentor was replaced with fresh sterile medium. The reactor was then operated at the fed-batch mode by pulse feeding concentrated substrate solution when the sugar level in the fermentation broth was close to zero. To evaluate the maximum butyric acid concentration achievable in the fermentation, the feeding was continued until the fermentation ceased to produce butyrate due to product inhibition. Samples were taken at regular intervals for the analysis of cell, substrate and product concentrations. The fermentation kinetics of xylose, the glucose/xylose mixture, and the CFH/CSL mixture were studied in the same reactor in the order stated, and glucose fermentation was studied in a second reactor.

Analytical Methods. Cell density was analyzed by measuring the optical density of the cell suspension at a wavelength of 620 nm (OD₆₂₀) with a spectrophotometer. A high performance liquid chromatography (HPLC) system was used to analyze the organic compounds, including glucose, xylose, fructose, arabinose, lactate, butyrate, and acetate in the fermentation broth and corn fiber hydrolysate. Gas production, including hydrogen and carbon dioxide, was monitored with a gas analyzer.

Fermentation of Glucose and Xylose. Fed-batch fermentations of glucose, xylose, and their mixture as the major carbon source by C. tyrobutyricum were studied at pH 6.0, and the results showed that both glucose and xylose were readily fermented to produce butyric and acetic acids. The fermentation reached a maximum butyrate concentration of 44.1 g/L from glucose and 37.3 g/L from xylose. In both fermentations, a similar butyrate yield (˜0.43 μg) was obtained. However, more cell growth and acetate production were obtained with glucose. The reactor productivity was also higher with glucose than with xylose (6.5 vs. 2.3 g/L·h). Based on the optical density in the fermentation broth, the specific growth rates (μ) for cells growing on glucose and xylose were found to be 0.113 and 0.058 h⁻¹, respectively.

In the fermentation of the xylose and glucose mixture, the bacterium metabolized glucose and xylose simultaneously, indicating no preference for either one of the two sugars as the carbon source. Glucose consumption was faster initially, but the consumption of xylose became faster after the first batch. In general, the fermentation kinetics with the glucose/xylose mixture were more similar to that for xylose fermentation, with an acetate/butyrate ratio of 0.13 μg. However, butyrate yield was slightly lower than that in the xylose fermentation, probably due to the higher biomass formation in the presence of glucose.

Fermentation of CFH and CSL. The feasibility of using CFH as the substrate for butyric acid fermentation was also studied. CFH, containing mainly carbon sources (glucose, xylose and arabinose), was supplemented with corn steep liquor (CSL), which provided the necessary nitrogen source for the bacterium. CSL also provided additional carbon sources as it contained high concentrations of glucose, lactate and fructose. The results showed that all the carbon sources (43.7 g/L) present in CFH and CSL were used in the fermentation. Arabinose appeared to be the most favored carbon source and was the first one consumed in the fermentation. It was followed by simultaneous consumption of glucose, xylose, and lactate. However, there were ˜2 g/L of xylose left at the end of fermentation. Also, fructose seemed to be the last carbon source used by C. tryrobutyricum. Compared with fermentations with xylose and glucose-xylose mixture as the substrates, both reactor productivity (2.91 g/L h) and butyric acid yield (0.474 μg based on the total carbon source consumed) were higher. The increased butyrate production could be attributed to additional nutrients present in CSL and reduced acetate formation. In general, the production of acetate was not as significant, probably because the high initial amount of acetate in the fermentation broth inhibited its formation. It can be concluded that CFH supplemented with CSL can be used efficiently as a substrate to produce butyric acid by C. tyrobutyricum.

### EXAMPLE 2

### Extractive Fermentation

Several varieties of anaerobic bacteria can produce butyric acid as a fermentation product from a wide range of substrates. Among them, Clostridium tyrobutyricum has excellent cell growth along with relatively high product purity and yield. However, butyric acid bacteria are inhibited by their acid products. Consequently, conventional butyric acid fermentation is usually limited by low reactor productivity, low product yield, and low final product concentration. Product recovery is therefore difficult and the process is uneconomical.

An integrated fermentation-separation process, such as extractive fermentation, can be used to reduce product inhibition and increase reactor productivity and product yield. Extractive fermentation may also allow the process to produce and recover the fermentation product in one continuous step, thus reducing downstream processing load and recovery costs. The advantages for extractive fermentation also include improved pH control in the reactor without the addition of base, as well as the ability to utilize a high-concentration substrate as the process feed.

In developing extractive fermentation for butyric acid production it is difficult to find a biocompatible solvent that also has a high extraction coefficient, or K_{eq} value, because solvents with high K_{eq} values are usually toxic to bacterial cells. It is also difficult to find an effective extractant at a pH value close to the optimal pH (usually ˜6 or higher) for fermentation. Most solvents work well only at a pH much lower than the pKₐ value of the organic acid to be extracted, and extraction efficiency decreases dramatically when the pH is higher than the pKₐ value.

An example of extractive fermentation for butyric acid production from glucose by immobilized cells of Clostridium tyrobutyricum in a fibrous bed bioreactor was executed by using 10% (v/v) Alamine 336 in oleyl alcohol as the extractant. The process was contained within a hollow-fiber membrane extractor to selectively remove butyric acid from the fermentation broth. The extractant was simultaneously regenerated by stripping with NaOH in a second membrane extractor. The fermentation pH was self-regulated by a balance between butyric acid production and removal of butyric acid by extraction, and was kept at ˜pH 5.5.

The extractive fermentation gave a much higher product concentration (>300 g/L) and product purity (91%) than conventional fermentation. Extractive fermentation also gave a higher reactor productivity (7.37 g/L·h) and butyric acid yield (0.45 μg). For comparison, the same fermentation without on-line extraction to remove butyric acid resulted in a final butyric acid concentration of ˜43.4 g/L, a butyric acid yield of 0.423 μg, and a reactor productivity of 6.77 g/L·h when the pH was 6.0. When the pH was 5.5, the final butyric acid concentration was 20.4 g/L, the butyric acid yield was 0.38 μg, and the reactor productivity was 5.11 g/L·h. The improved performance for the extractive fermentation can be attributed to reduced product inhibition by selectively removing butyric acid from the fermentation broth. Moreover, the solvent was not harmful to cells immobilized in the fibrous bed.

It can be concluded that butyric acid can be produced in an extractive fermentation process using an organic solvent for on-line separation of butyric acid from the fermentation broth. The butyric acid present in the extractant may be stripped by various methods, including stripping with a base solution (e.g., NaOH), a strong acid solution (e.g., HCl), or with hot water or steam. The butyric acid in the solvent also can be reacted directly with an alcohol to form an ester under the catalytic action of a lipase.

### EXAMPLE 3

### Esterification

An integrated fermentation, extraction, and esterification process (see FIG. 2) can be used to produce esters from alcohols and organic acids produced in fermentation. Butyric acid is first extracted into an amine solvent and then reacted with butanol to form butyl butyrate ester. In this process, the stripping (back extraction) step shown in FIG. 1 is replaced with esterification, with alcohol as the stripping solution and a catalyst to catalyze the reaction between alcohol and organic acids present in the extractant. The catalysts useful for esterification can be lipase, sulfuric acid and cation exchange resin (e.g., Amberlyst 15). However, lipase can catalyze the esterification reaction under mild conditions (lower temperature) and without any byproduct except for water as compared to organic synthesis. With proper control of the reaction medium, a high product yield of greater than 90% with close to 100% conversion can be obtained. The ester present in the amine solvent can be separated by distillation or other methods and the amine solvent can then be recycled back for use in the extraction process, as shown in FIG. 2.

In general, a solvent other than ethanol (e.g., n-hexane) is needed for lipase catalyst. However, organic acids in the low molecular-weight tertiary amine solvent (trialkyl amines) can be directly reacted with alcohols to produce esters. In fact, the esterification reaction is faster and more complete by reacting the organic acids and alcohols in the amine solvent, as compared with reaction in an aqueous solution. More than 90% conversion of the organic acid to its ester with an alcohol can be achieved with the reaction in the organic solvent. Also, the process can be operated continuously with a very steady product stream. In general, the esterification reaction is faster and more efficient with a higher molecular weight organic acid (i.e., butyric acid>propionic acid>lactic acid>acetic acid). Esterification of butyric acid with butanol present in an organic solvent such as Alamine 336 thus can be accomplished via the use of a lipase, preferably immobilized on a solid support. As compared to free lipase, immobilized lipase offers many benefits, including enzyme reuse, easy separation of product from enzyme and the potential to run continuous processes via packed-bed reactors. The stability of lipase is also improved. Immobilized lipase has a shift toward higher optimal temperature as compared to free lipase. Free lipase tends to aggregate in the presence of organic solvent, while immobilized lipase can provide good dispersion which results in a higher reaction rate. Immobilization by entrapment in gels can alleviate alcohol inhibition of enzyme activity for the production of ethyl butyrate. However, no alcohol inhibition was reported for the production of ethyl acetate.

Esterification is widely used for commercial production of ethyl lactate and other esters. Lipases have been successfully used as catalysts for the synthesis of esters on the industrial scale. The mild reaction conditions in the enzymatic reactions make it possible to obtain products of very high purity. An ethyl acetate yield of 93.2% can be achieved in 24 hours by free lipase catalysis and the lipase can be reused for more than 10 cycles. An ethyl butyrate yield of 93.3% can be achieved in 16 hours. Esterification of geraniol with acetic acid in n-hexane catalyzed by free lipase at 30° C. reached a conversion of 100% in 8 hours. For the esterification of lactic acid and ethanol catalyzed by free lipase at 30° C., 100% conversion can be achieved in 24 hours. Thus, organic acids can react with alcohols catalyzed by lipase to form esters and water. Removal of some water during the chemical reaction can improve reaction rate and conversion. Although a small amount of water is necessary for the lipase to maintain its optimal conformation in organic solvent and to have the optimal catalytic activity, too much water around the lipase can reverse the reaction.

Conventional esterification is a reversible reaction, and suffers from equilibrium limitation with less than 100% conversion. Removal of water during the chemical reaction can improve reaction rate and conversion. Water removal can be done by adsorption (with zeolite, celite, etc.), pervaporation, azeotropic distillation, nitrogen stripping, and vacuum. For nitrogen stripping and vacuum methods, alcohol must be added periodically to the reactor to compensate the loss of substrate due to evaporation. The water content of reaction mixtures can be controlled by Karl-Fischer titration to keep its level between 0.4-0.6% (w/w) in the reaction. Salt hydrates can also be used to control the water content.

Catalytic Hydrogenation of Butyric Acid

Hydrogenation of carboxylic acids to corresponding alcohols involves the activation of a carbonyl bond and addition of hydrogen to it. The carbonyl group is very stable and difficult to activate, so it normally requires expensive hydride agents such as LiAlH₄ in order to reduce carboxylic acids to alcohols. These reactions are not economical and not suitable for industrial use. However, carboxylic acids can also be catalytically converted to corresponding alcohols by hydrogenation with various catalysts under elevated pressures and temperatures. Besides alcohol, the hydrogenation reaction may also produce corresponding esters as by-products.

Industrial applications for carboxylic acid hydrogenation started as early as the 1930s. But this process is limited to fatty acids and fatty acid esters. Commercial hydrogenation catalysts currently employed by the industry are Cu/ZnO and Cu/Cr formulations. The general operating conditions for these catalysts are 200-300 atm hydrogen pressure and 150-250° C. The hydrogenation reaction requires high pressures and high temperatures to achieve acceptable productivity because it is difficult to activate the carbonyl group in the carboxylic acid. Furthermore, the copper catalyst is vulnerable to acids, which leads to metal ion loss and final product contamination. Additionally, in the presence of acids, the catalysts may be transformed to an inactive form. These disadvantages have restricted the use of the current commercial catalysts and promoted the search for new catalysts.

New research has identified highly active, stable, and selective catalysts for the hydrogenation of butyric acid. The preferred operation conditions are low pressure and low energy consumption. Several promising catalysts have been reported recently. Using a bimetallic catalyst consisting of Group VIII late transition-metal compounds and supported by Al₂O₃ or carbon, a high productivity (>95%) is achieved without ester formation at 180° C. and 100 atm hydrogen pressure. Another widely investigated catalytic system is Ru—Sn. With the Ru— Sn/Al₂O₃ system, 90% conversion with 100% selectivity was obtained at 260° C. and 98 atm hydrogen pressure. A MgO-NH₂—Ru catalytic system also showed a high activity for carboxylic acid hydrogenation, achieving 100% alcohol yield at 240° C. and 50 atm hydrogen pressure.

Compared to other catalytic systems, MgO-NH₂—Ru is relatively easy to prepare. MgO-NH₂—Ru catalyst is prepared in a two-step synthesis process. The first step is to prepare magnesia-supported poly-γ-aminopropylsiloxane by magnesia reacting with γ-aminopropyltriethoxysilane in a toluene solution. The solution is refluxed for one hour and then solvent is removed under reduced pressure to obtain a white powder (MgO-NH₂). The second step is to synthesize ruthenium complex, which adds RuCl₃ to MgO-NH₂ ethanol solution. The mixture is refluxed overnight to yield black MgO-NH₂—Ru. In the hydrogenation reaction, the catalyst is mixed with butyric acid solution (up to 30%) in a reactor, which is then pressurized with hydrogen to a desired pressure between 50 and 200 atm and heated to a predetermined temperature between 160 and 300° C. The reaction is allowed to continue for up to ˜20 hours until the conversion is near completion, and the product is analyzed with a gas chromatograph.

Group VIIIB compounds are excellent hydrogenation catalysts. However, ruthenium used in the MgO-NH₂—Ru catalyst is a precious metal. Using a non-precious metal, such as iron, cobalt and nickel, as a substitute for ruthenium can reduce the cost for the catalyst. Iron, cobalt and nickel are in the same transition metal group (Group VIIIB) as ruthenium, palladium and platinum. Raney Ni is a well known hydrogenation catalyst. Ni/Al₂O₃ and Ni/SiO₂ are used in industry for aldehydes/ketones, olefin, and phenol hydrogenation. Recently, an iron catalyst was developed for hydrogenation of ketones. All of these demonstrated that Fe, Co, and Ni based catalysts are useful for hydrogenation. Iron is more similar to ruthenium and has the greatest potential to replace ruthenium. Therefore, RuCl₃ can be replaced with FeCl₃, CoCl₂ or NiCl₂ in the catalyst synthesis. Different catalyst supporters, including MgO, SiO₂, Al₂O₃, active carbon, and molecular sieves may exhibit different effects on the reaction and catalyst performance. Also, it may be desirable to use bimetallic or even tri-metallic catalysts, such as Fe—Ni, Fe—Ni—Pd, for the hydrogenation reaction.

Ni—Pd catalyst supported by specially synthesized ZSM-5 (Zeolite Series of Mobile-5) can convert carboxylic acids to corresponding alcohols at mild conditions. However, it requires a large amount of energy to prepare ZSM-5. Alternatively, amorphous alloy, which has similar physical properties as ZSM-5 such as high surface area but does not require calcination, can be used as catalyst supports. Therefore, Ni, Fe, and Ni—Fe on amorphous alloy may be the preferred catalysts for carboxylic acid hydrogenation.

Traditional hydrogenation processes employ gas phase reactions. The advantages of gas phase reactions are that acids can be fully mixed with hydrogen for reaction. The metal loss for the catalysts is also relatively insignificant in a gas-phase reaction. However, gas-phase reactions are relatively difficult to operate because additional equipment and higher temperatures and pressure are required. The energy consumption is also significantly increased due to the vaporization of the acid and cooling the final products to the liquid state. Therefore, most of the recent studies of catalytic hydrogenation were carried out as the liquid-phase reaction, which has the advantage of not converting the non- or low-volatile carboxylic acids, such as butyric acid (b.p. 163.5° C. at 1 atm), to the gas phase before introducing to the reactor. Liquid-phase reaction is thus easier to operate and can save energy. However, hydrogen has a low solubility in water and the amount of hydrogen dissolved in the acid solution is limited, which constrains the contact among catalysts, acid, and hydrogen, and reduces productivity. Liquid-phase reaction can also cause significant metal loss due to the direct contact of acid solution with the catalysts, although this problem can be minimized with improved catalysts. A non-polar solvent, such as hexane, can be used to substitute water in the liquid-phase reaction. This can minimize the proton ions in the liquid phase and thus reduce the leakage of metal ions from the catalysts.

The low hydrogen solubility in the liquid substrate is one reason for the relatively low activity of carboxylic acid hydrogenation in a multi-phase heterogeneous system. One solution for this problem is to use supercritical fluid such as supercritical carbon dioxide and supercritical propane. A supercritical single-phase may be formed by adding supercritical fluid to reaction mixture. Through that, excess hydrogen is available for the reaction, resulting in high conversion rate (100%) and relatively high alcohol selectivity (60%-90%).

Most of the known catalysts are effective with long-chain and medium-chain fatty acids (with more than 5 to 10 carbons). In general, the hydrogenation reaction works faster and gives a higher yield when long- and medium-chain fatty acids are used. For short-chain fatty acids including carboxylic acids such as acetic, propionic, lactic, and butyric acids, the reaction is usually slower. In general, catalytic hydrogenation can give a high alcohol yield of more than 95%. The productivity ranges from ˜1 g/L·h for C3 carboxylic acid to ˜89 g/L·h for C8 fatty acid in the liquid-phase reaction systems. Butyric acid can be effectively converted to n-butanol with a high yield in the presence of hydrogen and catalysts via catalytic hydrogenation. Although butyl butyrate ester may be a byproduct of the reaction, its production is minimized in the presence of water, which induces hydrogenolysis or the hydrolysis of ester to its component alcohol and acid, which simultaneously undergoes hydrogenation to form alcohol.

It is difficult to activate the carbonyl group in the carboxylic acid, which limits the subsequent hydrogenation reaction and thus a good catalyst is usually required to enable the reaction. Alternatively, the carbonyl group of the carboxylic acid can be activated by forming an ester with an alcohol. The ester then undergoes hydrogenolysis in the presence of hydrogen and is broken down to form two alcohols. Therefore, butanol is produced from butyric acid by converting to butyl butyrate ester first. Esterification can be accomplished with either the acid or its ammonia salt in the presence of an acid catalyst or enzyme (lipase).

The fermentation-hydrogenation process provides overall butanol yields of ˜0.4 μg glucose, which is much higher than 0.15 μg to 0.25 μg obtained in the ABE fermentation.

A few of the many advantages of this novel process include:

Higher butanol yield from the biomass (sugar)-0.4 μg vs. 0.15 to 0.25 μg from ABE fermentation;

Higher productivity—butyric acid fermentation has a much higher productivity (>2 g/Lh) than ABE fermentation (usually much less than 0.5 g/Lh). Hydrogenation is a much faster chemical reaction (completion in less than a few hours) than ABE fermentation (requires more than 24-48 hours);

Higher butanol concentration—hydrogenation produces butanol at a much higher concentration, while ABE fermentation is limited to less than 2% due to the strong butanol inhibition to the microorganism. The higher butanol concentration from hydrogenation allows for economical recovery and purification of butanol.

Butanol is the only major product from the present process, while ABE fermentation produces acetone, butanol, and ethanol in a mixture that is more difficult to separate and purify. With the present process, biobutanol can be more economically produced from fermentable sugars present in abundant low-cost biomass. This provides an alternative biofuel that has more desirable properties than ethanol and can replace gasoline as a transportation fuel without affecting current infrastructure (pipeline, fuel station, and automobile).

It is noted that terms like “preferably,” “generally”, “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A method of producing butanol comprising:
providing a feedstock comprising a carbohydrate source;
fermenting the carbohydrate source in the presence of butyric-acid producing bacteria to produce a fermentation output comprising butyric acid and hydrogen; and
hydrogenating the butyric acid in the presence of a catalyst to produce butanol.

2. The method of claim 1, wherein the hydrogen produced by fermenting the carbohydrate source is employed in hydrogenating the butyric acid.

3. The method of claim 2, wherein the hydrogen produced by fermenting the carbohydrate source is the sole source of hydrogen employed in hydrogenating the butyric acid.

4. The method of claim 1, wherein the butyric-acid producing bacteria comprises at least one of Clostridium tyrobutyricum, Clostridium butyricum, Clostridium beijerinckii, Clostridium populeti and Clostridium thermobutyricum.

5. The method of claim 1, wherein the carbohydrate source comprises at least one of a biomass and food processing byproduct.

6. The method of claim 5, wherein the food processing byproduct comprises corn fiber hydrolysate.

7. The method of claim 5, wherein the biomass comprises glucose and pentose sugars.

8. The method of claim 1, wherein the catalyst comprises a metal oxide.

9. The method of claim 1, wherein the catalyst comprises a bimetallic catalyst comprising Group VIII late transition-metal compounds supported by Al₂O₃.

10. The method of claim 9, wherein the catalyst comprises Ru—Sn/Al₂O₃.

11. The method of claim 1, wherein the catalyst comprises a tri-metallic catalyst.

12. The method of claim 1, wherein the catalyst comprises MgO-NH₂—Ru.

13. The method of claim 1, wherein the catalyst comprises at least one of a copper/zinc chromite-based catalyst, a ruthenium-dioxide and a ruthenium-carbon.

14. The method of claim 1, wherein the catalyst comprises at least one of Ni, Fe and Ni—Fe on at least one of an amorphous alloy, zeolite and SiO₂.

15. A method of producing butanol comprising:
providing a feedstock comprising a carbohydrate source;
fermenting the carbohydrate source in the presence of Clostridium tyrobutyricum to produce a fermentation output comprising butyric acid and hydrogen; and
hydrogenating the butyric acid in the presence of a catalyst to produce butanol, wherein the hydrogen produced by fermenting the carbohydrate source is employed in hydrogenating the butyric acid.

16. The method of claim 15, wherein the hydrogen produced by fermenting the carbohydrate source is the sole source of hydrogen employed in hydrogenating the butyric acid.

17. The method of claim 15, wherein the method yields at least about 0.3 gram of butanol per gram of carbohydrate source.

18. A method of producing butanol comprising:
providing a feedstock comprising corn fiber hydrolysate;
fermenting the corn fiber hydrolysate in the presence of Clostridium tyrobutyricum to produce a fermentation output comprising butyric acid and hydrogen; and
hydrogenating the butyric acid in the presence of a catalyst to produce butanol, wherein the hydrogen produced by fermenting the corn fiber hydrolysate is employed in hydrogenating the butyric acid.

19. The method of claim 18, wherein the feedstock further comprises corn steep liquor.
